(19) United States
(12) Patent Application Publication
(10) Pub. No.: US 2016/0266066 A1
(43) Pub. Date: Sep. 15, 2016

(54) Screening Methods for Antiperspirants

(71) Applicant: COLGATE-PALMOLIVE COMPANY, New York, NY (US)

(72) Inventors: Long Pan, Cherry Hill, NJ (US); Shaotang Yuan, East Brunswick, NJ (US); Iraklis Pappas, Pennsauken, NJ (US); John Vaughn, Fair Haven, NJ (US); James G. Masters, Ringoes, NJ (US)

(73) Assignee: Colgata-Palmolive Company, New York, NY (US)

(21) Appl. No.: 15/036,261

(22) PCT Filed: Nov. 12, 2013
(86) PCT No.: PCT/US2013/069653

§ 371 Date: May 12, 2016

(51) Int. Cl. G01N 27/447; A61K 8/28; A61Q 15/00; A61K 8/19; A61K 8/44; A61K 8/26; A61K 8/27

(52) U.S. Cl. CPC G01N 27/44782; A61K 8/26; A61K 8/28; A61K 8/27; A61K 8/19; A61K 8/44; A61Q 15/00

(57) ABSTRACT

Methods of estimating the suitability of a candidate antiperspirant material by measuring the zeta potential of the material.

### BACKGROUND

The present invention relates to the measurement of zeta potential of putative antiperspirant active compounds to screen and determine likely effectiveness.

The function of antiperspirant (AP) is to prevent the sweat from coming out the skin. Two mechanisms of antiperspirant action are proposed: (i) AP metal salts combine with proteins in the sweat to form flocculant which blocks the sweat glands and (ii) AP metal salts hydrolyze in the presence of sweat to form metal hydroxide agglomerates that physically plug the sweat ducts. These salts are also typically quite acidic, and so reduce odor-causing bacteria, thereby providing a deodorant effect in addition to the antiperspirant effect.

Antiperspirant compositions typically contain aluminum chlorohydrate salts (ACH) or aluminum-zirconium glycine complex salts (ZAG). These salts tend to polymerize in solution, forming species with molecular weights ranging from about 500 to about 500,000 g/mol. In general, lower molecular weight species have greater antiperspirant effect than higher molecular weight species. It has been suggested that the smaller molecules more readily and more effectively enter sweat pores to occlude sweat pores, thereby producing the desired antiperspirant effect.

Current physical/chemical screens to identify optimal antiperspirant formulations have therefore focused on measuring the extent of antiperspirant polymerization by size exclusion chromatography (SEC), also known as gel filtration chromatography (GFC). Passage of small molecules through an SEC column is retarded while large molecules pass through more rapidly. Elution time of a polymeric substance migrating through the SEC column is correlated with the size of the polymer molecules, and this relationship allows the apparent molecular weight of a polymer to be determined. In most cases, 4 to 6 well defined groups of polymerized species in aluminum or aluminum-zirconium salt compositions can be identified by SEC and are known in the art as peaks 1, 2 (2 is not always present in the case of AIZr salts), 3, 4, 5, and 6 (6 is not always present) with the earlier peaks (1, 2, and 3) corresponding to the larger species and the later peaks (4, 5 and 6) corresponding to the smaller and more desirable species. Peaks correlate with weight average values of molecular weight for polymers, not as discrete values.

While size exclusion techniques are valuable, however, every commercial AP salt is composed of various species with different particle size showing multiple peaks in SEC profile. In addition, there are several species can also be detected even under one elution peak, say peak 4. The particle size as criteria to evaluate efficacy of commercial AP salts has certain limitations. Moreover, screening based exclusively on such techniques may be less suitable for determining the effectiveness of other active agents, as well as the effect of formulation conditions such as pH on the efficacy of the agents. There is thus a need for improved methods of screening, identifying and characterizing potential antiperspirant and/or deodorant compositions.

### BRIEF SUMMARY

To accurately identify efficacy of AP salts prior to clinical studies is exceptionally challenging, because of the high cost and long period required for the studies to be completed. Aluminum-based salts, including aluminum chlorohydrate (ACH), and aluminum zirconium chlorohydrate glycine complex (ZAG) are the two primary active ingredients in current antiperspirant products on the market. The mechanism of AP salts stop sweating involves the formation of precipitate to block sweat gland. The amount of precipitate formed in this process is related to the amount of sweat that can come out the skin. As proteins are one of the important components of human sweat, the interaction and formation of floc between metal cations, such as Al³⁺ and Zr⁴⁺, and biomolecules, such as proteins, cannot be neglected. While common characterization methods (SEC, NMR, DLS) do not provide any information on the flocculation process, we have determined that rapid flocculation of AP salts with the presence of proteins is a significant contributor to efficacy. As a result, we have determined that measuring zeta potential under conditions similar to use conditions is a powerful approach to estimate the efficacy of charged colloidal active candidates.

Zeta potential refers to the electrokinetic potential in colloidal systems. Specifically, the zeta potential is the electric potential in the interfacial double layer at the location of the slipping plane versus a point in the bulk fluid away from the interface. The zeta potential is the potential difference between the dispersion medium and the stationary layer of fluid attached to the dispersed particle.

The zeta potential correlates with the degree of repulsion between adjacent, similarly charged particles in a dispersion. For molecules and particles that are small enough to be significantly influenced by van der Waals forces, a large zeta potential will tend to confer stability, i.e., the particles will tend to repel one another, and the solution or dispersion will resist aggregation. When the zeta potential is small, however, attraction exceeds repulsion, and the dispersion will break and flocculate. The large or small zeta potential correlating with stability or aggregation respectively may be in large or small in a negative or positive direction—unless specifically identified by + or − sign, zeta potential values referred to herein are discussed in terms of absolute value; that is, unless specifically identified otherwise, “higher” refers to a zeta potential that is farther from zero, in either the positive or negative direction, while “lower” refers to a zeta potential that is closer to zero.

Here, it is desirable to have a zeta potential that is high enough in formulation to deter aggregation, but low enough to allow rapid flocculation and blockage of the pores. Because the zeta potential is strongly influenced by pH and by the presence of other charged molecules, we can evaluate both the zeta potential in formulation, and also in a more dilute solution (as will arise when the user perspires) and in the presence of proteins, etc. as would be expected to be found at the sweat pores.

The invention thus provides, in one embodiment, a method of estimating the suitability of a candidate antiperspirant material (which may be a single compound or combination of compounds) comprising measuring the zeta potential of a composition comprising the candidate antiperspirant material and a protein, and selecting candidates that provide a lower zeta potential than other candidates at the same relative concentration of candidate antiperspirant material and protein, or to put it another way, candidates that provide a zeta potential of zero at a lower concentration.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

Provided is a method of estimating the suitability of a candidate antiperspirant material comprising measuring the zeta potential of a composition comprising the candidate antiperspirant material and a protein (Method 1), e.g.,
1.1. Method 1 wherein the zeta potential is measured at different concentrations of the candidate antiperspirant material.
1.2. Any of the foregoing methods wherein the concentration of the candidate antiperspirant material at which the zeta potential is zero is determined.
1.3. Any of the foregoing methods comprising measuring the zeta potential provided by more than one candidate antiperspirant material, and selecting candidate antiperspirant material that provides a lower zeta potential than other candidates at the same concentration.
1.4. Any of the foregoing methods wherein the zeta potential is determined under conditions which are similar in one or more respects to use conditions.
1.5. Any of the foregoing methods wherein the zeta potential is determined under conditions similar to formulation conditions.
1.6. Any of the foregoing methods wherein the zeta potential is measured at different concentrations of the candidate antiperspirant material.
1.7. Any of the foregoing methods wherein the zeta potential is measured at different concentrations of the protein.
1.8. Any of the foregoing methods wherein the zeta potential of a composition having a fixed concentration of candidate antiperspirant material and of protein is measured and compared to a reference standard.
1.9. Any of the foregoing methods wherein a concentration of the candidate antiperspirant material and of the protein at which the zeta potential is zero is determined.
1.10. Any of the foregoing methods comprising measuring the zeta potential of more than one candidate antiperspirant material, and selecting the candidate antiperspirant material that provides a lower zeta potential than other candidates at the same concentration.
1.11. Any of the foregoing methods comprising
   providing a first composition comprising (i) a first candidate antiperspirant material and (ii) a carrier comprising a protein and a carrier solvent;
   measuring zeta potential of the first composition at different concentrations of the first candidate antiperspirant material and a fixed concentration of the protein; and
   determining a first concentration of the first candidate antiperspirant material that provides a zeta potential of zero.
1.12. The method of 1.11 further comprising
   providing a second composition comprising a second candidate antiperspirant material and a carrier of the second composition comprising a protein and a carrier solvent;
   measuring zeta potential of the second composition at different concentrations of the second candidate antiperspirant material and a fixed concentration of the protein, and
   determining a second concentration of the second candidate antiperspirant material that provides a zeta potential of zero,
   determining the lesser concentration between the first concentration and the second concentration, and
   selecting the candidate antiperspirant material that has a lower amount of antiperspirant material that provides a zeta potential of zero as having a better potential efficacy as an antiperspirant material.
1.13. Any of the foregoing methods of 1.1 to 1.10 comprising
   providing a first composition comprising (i) a first candidate antiperspirant material and (ii) a carrier comprising a protein and a carrier solvent;
   measuring zeta potential of the first composition at different concentrations of the protein and a fixed concentration of the first candidate antiperspirant material; and determining a first concentration of the protein that provides a zeta potential of zero.
1.14. The method of 1.13 further comprising
   providing a second composition comprising a second candidate antiperspirant material and a carrier of the second composition comprising a protein and a carrier solvent;
   measuring zeta potential of the second composition at different concentrations of the protein and at a fixed concentration of the second candidate antiperspirant material, and
   determining a second concentration of the protein that provides a zeta potential of zero,
   determining the lesser concentration between the first concentration and the second concentration, and
   selecting the candidate antiperspirant material that has a lower amount of protein that provides a zeta potential of zero as having a better potential efficacy as an antiperspirant material.
1.15. Any foregoing method wherein the zeta potential of the candidate antiperspirant material is determined under conditions which are similar in one or more respects to use conditions, and optionally also determined under conditions similar to formulation conditions.
1.16. Any foregoing method wherein a candidate material is selected for further evaluation when the concentration of the selected candidate material at which the zeta potential is zero is lower than the concentration of other candidate materials at which the zeta potential is zero.
1.17. Any foregoing method wherein the candidate antiperspirant material comprises one or more salts selected from an aluminum salt, a zirconium salt, an aluminum-zirconium salt, a zinc salt, a copper salt, and amino acid complexes with any of the foregoing.
1.18. Any foregoing method wherein the protein is negatively charged in aqueous solution at pH 7.
1.19. Any foregoing methods wherein the protein exhibits a zeta potential in distilled water of −10 to −40 mV at a concentration of 1-20 mg/ml, for example −10 to −20 mV at a concentration of 20 mg/ml.
1.20. Any foregoing methods wherein the protein is bovine serum albumin.
1.21. Any foregoing methods wherein the zeta potential provided by positive control antiperspirant material having known antiperspirant efficacy is also measured.
1.22. Any of the foregoing methods wherein the zeta potential of the candidate material is measured at pH 5-8.
1.23. Any of the foregoing methods wherein the zeta potential of the candidate material is measured at pH 3-5 and also at a higher pH 5-11, e.g., 5-7, wherein the zeta potential provided by the candidate material at the same concentration is closer to zero at the higher pH than at the lower pH.
1.24. Any of the foregoing methods wherein the candidate material is selected for further evaluation when the concentration of the selected candidate material at which the zeta potential is zero is lower than the concentration of other candidate materials at which the zeta potential is zero.
1.25. Any of the foregoing methods wherein a selected candidate material has a zeta potential <20 mV in the presence of protein, and optionally further that have a higher zeta potential under formulation conditions, e.g., >30 mV.
1.26. Any of the foregoing methods wherein the candidate antiperspirant material comprises one or more salts selected from a metal salt, for example, an aluminum salt, a zirconium salt, an aluminum-zirconium salt, a zinc salt, a copper salt, and combinations thereof; for example wherein the candidate antiperspirant material comprises a salt selected from one or more of an aluminum chlorohydrate (ACH), an aluminum zirconium chlorohydrate glycine complex (ZAG), a zirconium chlorohydrate, and combinations thereof.
1.27. Any of the foregoing methods wherein the concentration of protein is between 1-50 mg/ml, e.g., 5-25 mg/ml.
1.28. Any of the foregoing methods wherein the zeta potential provided by a positive control antiperspirant material having known antiperspirant efficacy is also measured.
1.29. Any of the foregoing methods wherein the candidate material is selected on the basis of having lower zeta potential under use conditions, e.g., under conditions wherein protein is present and/or the pH is 5-7, compared to formulation conditions, e.g., wherein protein is not present and/or the pH is different from the pH under use conditions, e.g., <pH 5 or >pH 7.
1.30. Any of the foregoing methods wherein the candidate material is selected on the basis of having lower zeta potential under use conditions, e.g., under conditions wherein protein is present under use conditions, and a higher zeta potential under formulation conditions.

In any of the methods when measuring at different concentrations, prior tested samples can be used to prepare the next sample for testing by adding more of the antiperspirant/protein to the composition to form a new concentration.

Also provided is an antiperspirant salt identified or selected on the basis of a method of the invention, e.g., on the basis of any of Method 1. et seq.

In another embodiment, provided is the use of a zeta potential analyzer to screen or compare materials for possible antiperspirant efficacy, e.g., in a method according to any of Method 1, et seq.

In another embodiment, provided is the use of a protein, e.g., a negatively charged protein, e.g., bovine serum albumin, in an assay to predict the efficacy of a candidate antiperspirant active material, e.g., in a method according to any of Method 1, et seq.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Example 1

### Measuring Zeta Potential of Different Antiperspirant Salts in the Presence of Protein

Experiments are designed to explore and validate using Zeta-Potential (ZP) measurement as a way to pre-screen the efficacy of antiperspirant (AP) salts. In order to study the interaction of AP salts with the proteins from sweat and the efficacy of different types of AP salts, an experiment is performed mixing AP salts with BSA and measuring the ZP. The ZP measurement of AP-BSA mixture solution is used to determine the amount of AP salts required to contribute a zero ZP to the solution. The lower amount of AP salt used, the more efficacious the salt would be. The experimental results are correlated with results reported from clinical studies. The correlation demonstrates that ZP measurement of AP-proteins mixture solutions can be used to evaluate the efficacy of AP salts.

20 mg/ml of BSA solution is prepared by dissolving 40 g of solid BSA in 2000.0 ml of deionized water (DIW) under vigorously stirring to form transparent solution. AP-BSA mixture solutions are prepared by adding varying amount of AP powder into 18 ml of above BSA solutions. The zeta potential of pure BSA solution and AP-BSA mixture solutions are recorded by ZetaSizer Nano Series (Malvern Instruments). All AP-BSA mixture solutions are centrifuged at 5000 rpm for 30 minutes. 1 ml of supernatant is transferred into cuvette by disposable pipet. After being attached with the Universal Dip Cell (ZEN 1002, Malvern Instruments), the cell is placed into the Zetasizer for ZP measurement.

BSA used in this experiment is available from Sigma-Aldrich (St. Louis, Mo.). Aluminum Chlorohydrate (ACH), Activated ACH, Aluminum Sesquichlorohydrate (ASCH), Aluminum Zirconium Glycine (ZAG) and Activate ZAG are available from Summit (Huguenot, N.Y.).

Table 1 shows the change of zeta potential of solutions as the amount of antiperspirant salt is increased.

TABLE 1

| | ASCH | | | | | | | | | |
|---|---|---|---|---|---|---|---|---|---|---|
| Al salts | Amount | 0 | 10.8 | 14.4 | 18 | 21.6 | 22.5 | 25.2 | 28.8 | 54 |
| | added(mg) | | | | | | | | | |
| | Zeta | −16.88 | −13.34 | −10.23 | −7.85 | −1.94 | 0.21 | 5.76 | 7.38 | 14.56 |
| | potential(mV) | | | | | | | | | |
| | Activated ACH | | | | | | | | | |
| | Amount | 0 | 10.8 | 14.4 | 18 | 20.7 | 21.6 | 25.2 | 28.8 | 54 |
| | added(mg) | | | | | | | | | |
| | Zeta | −16.88 | −12.11 | −11.50 | −5.95 | −0.12 | 0.70 | 4.68 | 7.22 | 15.21 |
| | potential(mV) | | | | | | | | | |
| | ACH | | | | | | | | | |
| | Amount | 0 | 10.8 | 14.4 | 18 | 21.6 | 24.1 | 25.2 | 28.8 | 54 |
| | added(mg) | | | | | | | | | |
| | Zeta | −16.88 | −13.92 | −9.31 | −8.60 | −4.49 | 0.79 | 1.77 | 4.67 | 12.36 |
| | potential(mV) | | | | | | | | | |
| | ZAG | | | | | | | | | |
| Zr related | Amount | 0 | 10.8 | 14.4 | 18 | 21.6 | 25.2 | 28.8 | 54 | N/A |
| salts | added(mg) | | | | | | | | | |
| | Zeta | −16.88 | −11.90 | −11.52 | −9.63 | −6.30 | 0.42 | 6.34 | 18.10 | N/A |
| | potential(mV) | | | | | | | | | |
| | Activated ZAG | | | | | | | | | |
| | Amount | 0 | 10.8 | 14.4 | 18 | 21.6 | 22.7 | 25.2 | 28.8 | 54 |
| | added(mg) | | | | | | | | | |
| | Zeta | −16.88 | −11.50 | −10.36 | −7.80 | −4.08 | −0.74 | 4.19 | 10.04 | 20.60 |
| | potential(mV) | | | | | | | | | |
| | Zr-Glycine Complex | | | | | | | | | |
| | Amount | 0 | 10.8 | 14.4 | 16.2 | 18 | 21.6 | 25.2 | 28.8 | 54 |
| | added(mg) | | | | | | | | | |
| | Zeta | −16.88 | −6.49 | −0.66 | 4.12 | 9.04 | 12.52 | 18.46 | 20.96 | 31.42 |
| | potential(mV) | | | | | | | | | |
| | Al₁₃-mer | | | | | | | | | |
| Al | Amount | 0 | 10.8 | 14.4 | 18 | 21.6 | 25.2 | 28.8 | 32.4 | 54 |
| polycations | added(mg) | | | | | | | | | |
| | Zeta | −16.88 | −13.92 | −11.96 | −10.80 | −9.07 | −6.94 | −3.85 | −0.53 | 6.49 |
| | potential(mV) | | | | | | | | | |
| | Al₃₀-mer | | | | | | | | | |
| | Amount | 0 | 10.8 | 14.4 | 18 | 21.6 | 25.2 | 28.8 | 54 | N/A |
| | added(mg) | | | | | | | | | |
| | Zeta | −16.88 | −14.40 | −11.51 | −9.33 | −0.76 | 4.85 | 9.12 | 12.52 | N/A |
| | potential(mV) | | | | | | | | | |

The results in Table 1 are plotted and analyzed to determine the salt concentration where zeta potential is zero, and the salts are ranked accordingly. Table 2 summarizes the amount of salts required to change the zeta potential of BSA solution to (or close to) zero and the pH as this point. It clearly shows that the addition of the salts causes the zeta potential of BSA solution changes from negative to zero, and then to positive. At the point of zero zeta potential, optimal amount of floe is formed. By comparing the amount of salts required to move the zeta potential of the solution to zero, we can predict the antiperspirant efficacy of these salts:

TABLE 2

| | | pH at | Amount | Comparison of |
|---|---|---|---|---|
| | | zeta | of AP | efficacy predicted |
| | | potential | required | by zeta potential |
| samples | | is 0 | (mg) | technique |
| Al salts | ASCH | 5.25 | 20.7 | ASCH > Activated |
| | Activated ACH | 5.61 | 22.5 | ACH > ACH |
| | ACH | 5.28 | 24.0 | |
| Zr related | Activated ZAG | 5.28 | 22.7 | Zr-Gly > Activated |
| salts | ZAG | 5.65 | 25.2 | ZAG > ZAG |
| | Zr-Gly | 5.08 | 14.4 | |
| | complex | | | |
| Al | Al₁₃-mer | 5.58 | 32.4 | Al₃₀-mer > |
| polycations | Al₃₀-mer | 5.33 | 21.6 | Al₁₃-mer |

In Al active salts category, three commercial grade aluminum chlorohydrate salts are used: ASCH, Activated ACH and ACH. About 20.7 mg, 22.5 mg, and 24.0 mg of ASCH, Activated ACH and ACH, respectively, is needed to provide a zeta potential of zero to the solutions. Based on these results, it could be predicted that the ASCH would be the most efficacious in the Al active category, because it would be expected to flocculate at a lower concentration than the others in the presence of protein; Activated ACH would be the second most efficacious; and then the non-activated ACH would be the least efficacious. These results correlate well with the results of clinical studies.

In the second case, two commercial and one experimental zirconium salts are used. The results suggest that the amount of Activated ZAG required to reach a ZP value of zero and therefore flocculate is a lower amount than required for ZAG, which means Activated ZAG is more efficacious than ZAG. Again, the result matches the results in clinical studies. The third salt in this category is a zirconium-glycine complex molecule which contains Zr but not Al, which performs better than the two commercial ZrAl salts, making it a promising candidate for further development as AP active ingredient.

Studies from wastewater treatment have demonstrated that Al₃₀-mer, as an 18+ polycation, outperforms Al₁₃-mer (7+) in terms of coagulation/flocculation efficacy. In the third case, we predict the efficacy of two aluminum polyoxycations, Al₁₃-mer and Al₃₀-mer, with the zeta potential in the presence of protein being measured as a function of the amount of active in milligrams. Results clearly show that Al₃₀-mer performs better than Al₃-mer, which correlates to the results that has been found. The results from this experiment also suggests that the 30-mer would be expected to perform better than the 13-mer as AP active in clinical trials.

We claim:

1. A method of estimating the suitability of a candidate antiperspirant material comprising measuring the zeta potential of a composition comprising the candidate antiperspirant material and a protein.

2. The method of claim 1 wherein the zeta potential is measured at different concentrations of the candidate antiperspirant material.

3. The method of claim 1 wherein the zeta potential is measured at different concentrations of the protein.

4. The method of claim 1 wherein the zeta potential of a composition having a fixed concentration of candidate antiperspirant material and of protein is measured and compared to a reference standard.

5. The method of claim 1 wherein a concentration of the candidate antiperspirant material and of the protein at which the zeta potential is zero is determined.

6. The method of claim 1 comprising measuring the zeta potential of more than one candidate antiperspirant material, and selecting the candidate antiperspirant material that provides a lower zeta potential than other candidates at the same concentration.

7. The method of claim 1 comprising providing a first composition comprising (i) a first candidate antiperspirant material and (ii) a carrier comprising a protein and a carrier solvent;
measuring zeta potential of the first composition at different concentrations of the first candidate antiperspirant material and a fixed concentration of the protein; and
determining a first concentration of the first candidate antiperspirant material that provides a zeta potential of zero.

8. The method of claim 7 further comprising
providing a second composition comprising a second candidate antiperspirant material and a carrier of the second composition comprising a protein and a carrier solvent;
measuring zeta potential of the second composition at different concentrations of the second candidate antiperspirant material and a fixed concentration of the protein, and determining a second concentration of the second candidate antiperspirant material that provides a zeta potential of zero,
determining the lesser concentration between the first concentration and the second concentration, and
selecting the candidate antiperspirant material that has a lower amount of antiperspirant material that provides a zeta potential of zero as having a better potential efficacy as an antiperspirant material.

9. The method of claim 1 comprising providing a first composition comprising (i) a first candidate antiperspirant material and (ii) a carrier comprising a protein and a carrier solvent;
measuring zeta potential of the first composition at different concentrations of the protein and a fixed concentration of the first candidate antiperspirant material; and
determining a first concentration of the protein that provides a zeta potential of zero.

10. The method of claim 9 further comprising
providing a second composition comprising a second candidate antiperspirant material and a carrier of the second composition comprising a protein and a carrier solvent;
measuring zeta potential of the second composition at different concentrations of the protein and at a fixed concentration of the second candidate antiperspirant material, and determining a second concentration of the protein that provides a zeta potential of zero,
determining the lesser concentration between the first concentration and the second concentration, and
selecting the candidate antiperspirant material that has a lower amount of protein that provides a zeta potential of zero as having a better potential efficacy as an antiperspirant material.

11. The method of claim 1 wherein the zeta potential of the candidate antiperspirant material is determined under conditions which are similar in one or more respects to use conditions, and optionally also determined under conditions similar to formulation conditions.

12. The method of claim 1 wherein a candidate material is selected for further evaluation when the concentration of the selected candidate material at which the zeta potential is zero is lower than the concentration of other candidate materials at which the zeta potential is zero.

13. The method of claim 1 wherein the candidate antiperspirant material comprises one or more salts selected from an aluminum salt, a zirconium salt, an aluminum-zirconium salt, a zinc salt, a copper salt, and amino acid complexes with any of the foregoing.

14. The method of claim 1 wherein the protein is negatively charged in aqueous solution at pH 7.

15. The method of claim 1 wherein the protein provides a zeta potential in distilled water of −10 to −40 mV at a concentration of 1-20 mg/ml.

16. The method of claim 1 wherein the protein is bovine serum albumin.

17. The method of claim 1 wherein the zeta potential of positive control antiperspirant material having known antiperspirant efficacy is also measured.

18. (canceled)

19. (canceled)
